(19) United States
(12) Patent Application Publication
(10) Pub. No.: US 2025/0387615 A1
(43) Pub. Date: Dec. 25, 2025

(54) CONNECTOR, ELECTRICAL STIMULATION SYSTEM, AND ASSEMBLY METHOD OF ELECTRICAL STIMULATION SYSTEM

(71) Applicant: Coforce Medical Co., Ltd., Taipei City (TW)

(72) Inventors: Li-Wei Luo, New Taipei City (TW); Chia-Hsiung Peng, New Taipei City (TW)

(73) Assignee: Coforce Medical Co., Ltd., Taipei City (TW)

(21) Appl. No.: 18/967,526

(22) Filed: Dec. 3, 2024

(30) Foreign Application Priority Data
Jun. 19, 2024 (TW) 113122590

(51) Int. Cl. A61N 1/04

(52) U.S. Cl. CPC A61N 1/0488; A61N 1/0492

(57) ABSTRACT

A connector, an electrical stimulation system, and an assembly method of the electrical stimulation system are provided. The connector is adapted to fix a lead and to electrically connect the lead to an electrical stimulation device. The lead includes a filament and an insulation layer. The connector includes an upper cover, a base, an electrical pad, and a first fastener. The upper cover has a top portion. The base combined with the upper cover has a bottom portion. At least one of the upper cover and the base forms an opening. The electrical pad is disposed on the bottom portion. The first fastener is passed through the top potion and is locked toward a direction of the bottom portion. The lead is inserted into the connector from the opening. The first fastener moves along the direction toward the bottom portion to damage the insulation layer of the lead.

### CROSS-REFERENCE TO RELATED APPLICATION

This application claims the priority benefit of Taiwan application serial no. 113122590, filed on Jun. 19, 2024. The entirety of the above-mentioned patent application is hereby incorporated by reference herein and made a part of this specification.

### BACKGROUND

### Technical Field

The disclosure relates to a connector, an electrical stimulation system, and an assembly method of the electrical stimulation system.

### Description of Related Art

In recent years, with the development of precision manufacturing technology, the size of medical instruments has been miniaturized and convenient for surgery. For example, the medical instruments that can be applied to or implanted into the human body to perform medical treatments include electrical stimulation systems. Benefiting from the electrical stimulation systems, at least tens of thousands of people undergo surgeries using the electrical stimulation systems every year.

However, in the conventional electrical stimulation system, there is no good design that allows the user to quickly and conveniently fix the lead to the connector of the electrical stimulation system. Therefore, how to conveniently fix the lead to the connector of the electrical stimulation system is an urgent issue that needs to be solved.

### SUMMARY

The disclosure provides a connector with usage convenience.

The disclosure provides an electrical stimulation system with usage convenience.

The disclosure provides an assembly method of an electrical stimulation system that is convenient to assemble and disassemble.

A connector of the disclosure is adapted to fix a lead and to electrically connect the lead to an electrical stimulation device. The lead includes a filament and an insulation layer covering the filament. The connector includes an upper cover, a base, an electrical pad, and a first fastener. The upper cover has a top portion. The base combined with the upper cover has a bottom portion. At least one of the upper cover and the base forms an opening. The electrical pad is disposed on the bottom portion and is electrically connected to the electrical stimulation device. The first fastener is passed through the top portion and is fastened along a direction toward the bottom portion. When the lead is inserted into the connector from the opening, the first fastener moves along the direction toward the bottom portion to damage the insulation layer of the lead, so that the filament is electrically conductive to the electrical pad.

In an embodiment of the disclosure, the connector further includes at least one second fastener to fasten the upper cover and the base.

In an embodiment of the disclosure, the bottom portion has a lead accommodating groove exposed from a side surface of the base and having a recess.

In an embodiment of the disclosure, the upper cover also has a limiting portion. The limiting portion extends from the top portion along a first direction. The base has multiple limiting grooves located on two opposite sides, and the limiting grooves are combined with the corresponding limiting portions to form the opening with the recess.

In an embodiment of the disclosure, along an entry direction of the lead, a groove diameter of the lead accommodating groove varies.

In an embodiment of the disclosure, a bottom portion of the first fastener has a pressing surface, and a diameter of the pressing surface is greater than a winding diameter of the lead.

In an embodiment of the disclosure, the connector further includes at least two first positioning structures extending from two opposite sides of the top portion of the upper cover; and at least two second positioning structures respectively located on two opposite sides of the bottom portion. Each of the second positioning structures has a first engaging portion and a second engaging portion along a first direction, and the first engaging portion is located between the top portion and the second engaging portion. When the first positioning structure is combined with the first engaging portion, there is a gap between the upper cover and the base, and the upper cover and the base have a first relative position. When the first positioning structure is combined with the second engaging portion, the upper cover is combined with the base, and the upper cover and the base have a second relative position.

In an embodiment of the disclosure, the first positioning structure has two sets of hooks, and the two sets of hooks are formed respectively extending from a first side and a second side of the top portion toward the first direction. Hooking directions of the two sets of hooks are opposite.

In an embodiment of the disclosure, the top portion also has an abutting rib located between the first positioning structures, and when the first positioning structures are combined with the second engaging portions, the abutting rib abuts against the lead.

An electrical stimulation system of the disclosure includes a lead, an electrical stimulation device, and a connector. The lead includes a filament and an insulation layer covering the filament. The connector is adapted to fix the lead. The connector is electrically connected to the electrical stimulation device. The connector includes an upper cover, a base, an electrical pad, and a first fastener. The upper cover has a top portion. The base is combined with the upper cover and has a bottom portion. At least one of the upper cover and the base forms an opening. The lead is inserted into the connector from the opening. The electrical pad is disposed on the bottom portion and is electrically connected to the electrical stimulation device. When the first fastener is passed through the top portion and is fastened along a direction toward the bottom portion, the first fastener moves along the direction toward the bottom portion to damage the insulation layer of the lead, so that the filament is electrically conductive to the electrical pad.

In an embodiment of the disclosure, the connector further includes at least one second fastener to fasten the upper cover and the base.

In an embodiment of the disclosure, the connector further includes at least two first positioning structures extending from two opposite sides of the top portion of the upper cover; and at least two second positioning structures respectively located on two opposite sides of the bottom portion. Each of the second positioning structures has a first engaging portion and a second engaging portion along a first direction, and the first engaging portion is located between the top portion and the second engaging portion. When the first positioning structure is combined with the first engaging portion, there is a gap between the upper cover and the base, and the upper cover and the base have a first relative position. When the first positioning structure is combined with the second engaging portion, the upper cover is combined with the base, and the upper cover and the base have a second relative position.

In an embodiment of the disclosure, the electrical stimulation system further includes an unlocking member having at least one protruding portion. The second engaging portion is an open hole, and the protruding portion of the unlocking member cooperates with the open hole.

In an embodiment of the disclosure, the bottom portion has a lead accommodating groove exposed from a side surface of the base and having a recess.

In an embodiment of the disclosure, a bottom portion of the first fastener has a pressing surface, and a diameter of the pressing surface is greater than a winding diameter of the lead.

An assembly method of an electrical stimulation system of the disclosure at least includes the following steps. A lead is provided. The lead includes a filament and an insulation layer covering the filament. An electrical stimulation device is provided. A connector adapted to fix the lead is provided. The connector includes an upper cover, a base, an electrical pad, and a first fastener. The upper cover has a top portion. The base combined with the upper cover has a bottom portion. At least one of the upper cover and the base forms an opening. The lead is inserted into the connector from the opening. The electrical pad is disposed on the bottom portion and is electrically connected to the electrical stimulation device. When the first fastener is passed through the top portion and is fastened along a direction toward the bottom portion, the first fastener moves along the direction toward the bottom portion to damage the insulation layer of the lead, so that the filament is electrically conductive to the electrical pad, and the lead is electrically connected to the electrical stimulation device.

In an embodiment of the disclosure, the connector further includes at least two first positioning structures extending from two opposite sides of the top portion of the upper cover; and at least two second positioning structures respectively located on two opposite sides of the bottom portion. Each of the second positioning structures has a first engaging portion and a second engaging portion along a first direction. The first engaging portion is located between the top portion and the second engaging portion. The assembly method of the electrical stimulation system includes the following steps. The first positioning structure is combined with the first engaging portion. There is a gap between the upper cover and the base, and the upper cover and the base have a first relative position. The first positioning structure is combined with the second engaging portion. The upper cover covers the base, and the upper cover and the base have a second relative position.

In an embodiment of the disclosure, a protruding portion of an unlocking member is correspondingly inserted into the second engaging portion to release the first positioning structure and the second engaging portion from being combined.

In an embodiment of the disclosure, after releasing the first positioning structure and the second engaging portion from being combined, an elastic restoring force of the first positioning structure displaces the upper cover relative to the base along an opposite direction the first direction, and the first positioning structure is correspondingly snapped into the first engaging portion again.

Based on the above, in the connector, the electrical stimulation system using the connector, and the assembly method of the electrical stimulation system of the disclosure, the first fastener is passed through the upper cover and is fastened toward to the base to damage the insulation layer of the lead, so that the filament may be electrically conductive to the electrical pad of the connector via a simple fastening step, thereby enabling the lead to be electrically conductive to the electrical stimulation device.

### BRIEF DESCRIPTION OF THE DRAWINGS

FIG. 1 is a schematic view of an electrical stimulation system according to a first embodiment of the disclosure.

FIG. 2A is an exploded schematic view of a connector applied to the electrical stimulation system of FIG. 1.

FIG. 2B is a schematic view of an upper cover and a base having a first relative position after assembling the connector of FIG. 2A.

FIG. 2C is a cross-sectional view of a section A-A of FIG. 2B.

FIG. 2D is a schematic view of FIG. 2A from another perspective.

FIG. 3A is a schematic view of a base of FIG. 2A from a different perspective.

FIG. 3B is a schematic view of a variation of a lead accommodating groove of FIG. 3A.

FIG. 4 is a partially enlarged schematic view of the lead.

FIG. 5 is an assembly flowchart of the electrical stimulation system.

FIG. 6A is a schematic view of the lead fixed in the connector.

FIG. 6B is a schematic view of a first positioning structure combined with a second engaging portion.

FIG. 7 is a relationship diagram of a diameter of a pressing surface of a fastener and a winding diameter of the lead.

FIG. 8A to FIG. 8C are schematic views of damaging an insulation layer of the lead as the fastener is pressed downward, so that a filament is exposed and contacts an electrical pad.

FIG. 9A is a schematic view of using an unlocking member and the connector.

FIG. 9B is a schematic view of using the unlocking member to release the upper cover and the base of the connector from being combined.

FIG. 10A is an exploded schematic view of a connector of an electrical stimulation system according to a second embodiment of the disclosure.

FIG. 10B is a schematic view of a base in FIG. 10A from another perspective.

FIG. 11A is an assembly schematic view of a connector of FIG. 10A.

FIG. 11B is a cross-sectional view along a sectional line B-B of FIG. 11A.

FIG. 11C is a cross-sectional view along a sectional line C-C of FIG. 11A.

FIG. 12 is a schematic view of using a hand tool to adjust a position of a first fastener in a pillar hole.

### DESCRIPTION OF THE EMBODIMENTS

### First Embodiment

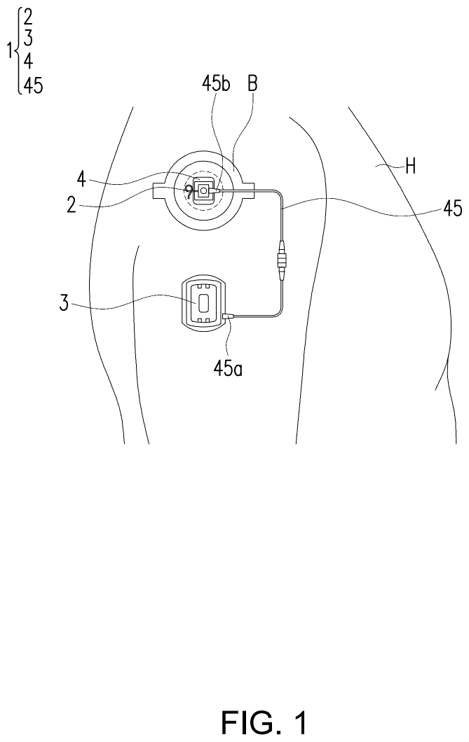

FIG. 1 is a schematic view of an electrical stimulation system according to a first embodiment of the disclosure. Please refer to FIG. 1. An electrical stimulation system 1 of the embodiment includes a lead 2, an electrical stimulation device 3, and a connector 4, wherein the connector 4 is used to fix the lead 2, so that the lead 2 is electrically connected to the electrical stimulation device 3 through the connector 4, and the connector 4 may be connected to the electrical stimulation device 3 through a cable 45, but not limited thereto.

In the embodiment, the electrical stimulation system 1 is a minimum-invasive system. The lead 2 has an electrode for electrical stimulation. One end of the lead is fixed and electrically connected to the connector 4, and the other end having the electrode may be coiled, linear, or hook-shaped and implanted and placed into a human body H or an animal body. In order to prevent the position of the electrode in the human body H from shifting after implantation, the lead 2 may also use an anchor or tines to assist in fixing the relative position of the lead 2 in the human body H, wherein the anchor or the tines may be separate elements or integrally formed with the lead 2. According to different electrical stimulation areas of the electrode on the lead 2, the electrical stimulation system 1 may also be divided into a central nerve electrical stimulation system and a peripheral nerve electrical stimulation system.

It should be noted that although the electrical stimulation system 1 of the embodiment takes a minimum-invasive peripheral nerve electrical stimulation system as an example, it is not limited thereto. In other embodiments, depending on whether the electrical stimulation system 1 is implanted into the body, the electrical stimulation system 1 may also be transcutaneous or implantable. Taking the transcutaneous electrical stimulation system 1 as an example, a patch (for example, a waterproof film B) is used to fix the connector 4 onto the skin surface of the human body H. In the implantable electrical stimulation system 1, after the implantable electrical stimulation device 1 is electrically connected to the lead 2 and the connector 4, the electrical stimulation device 1, the lead 2, and the connector 4 are implanted into the human body H together, and the implantable electrical stimulation system 1 and the connector 4 are not only connected by the cable 45, but may also be placed outside the human body H or worn on the surface of the human body H by being further equipped with an external controller (not shown), and activation control signals or electrical energy are provided to the implantable electrical stimulation device 1 for electrical stimulation through wireless control or power supply.

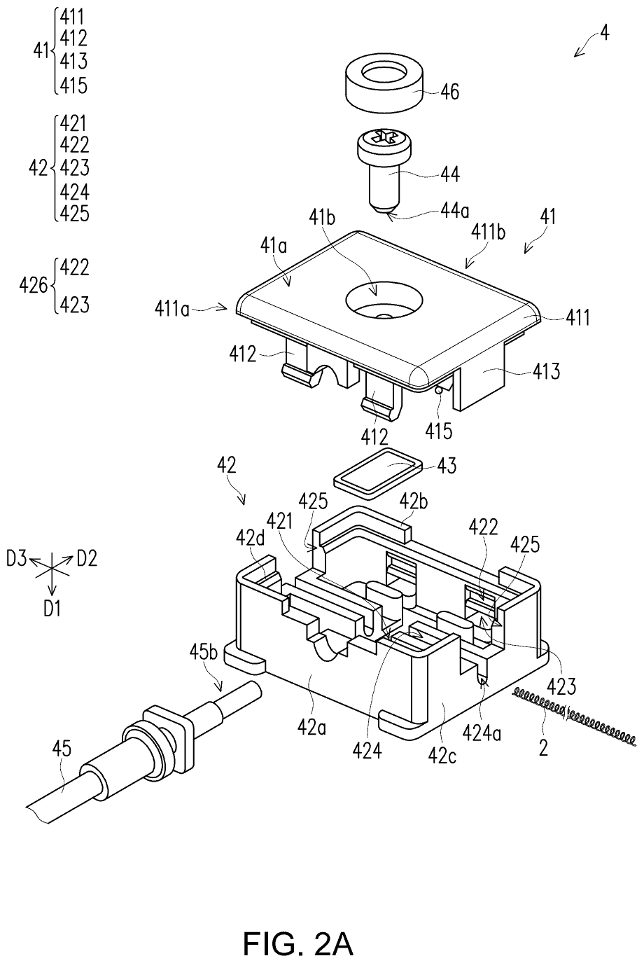

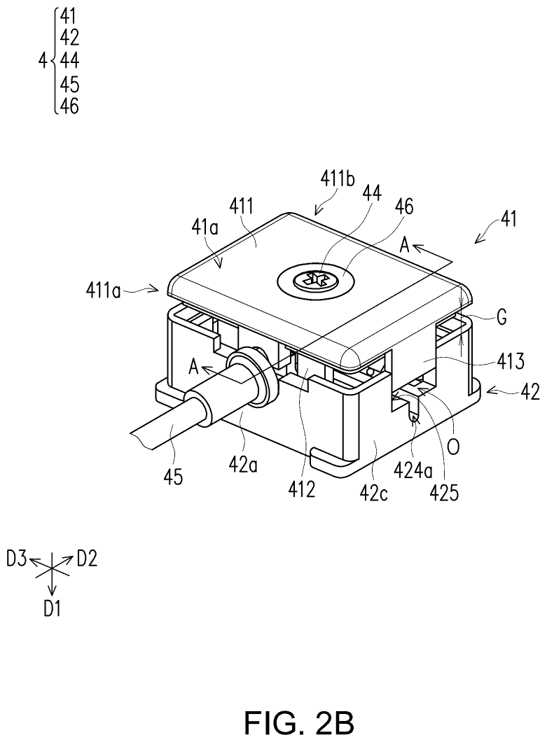

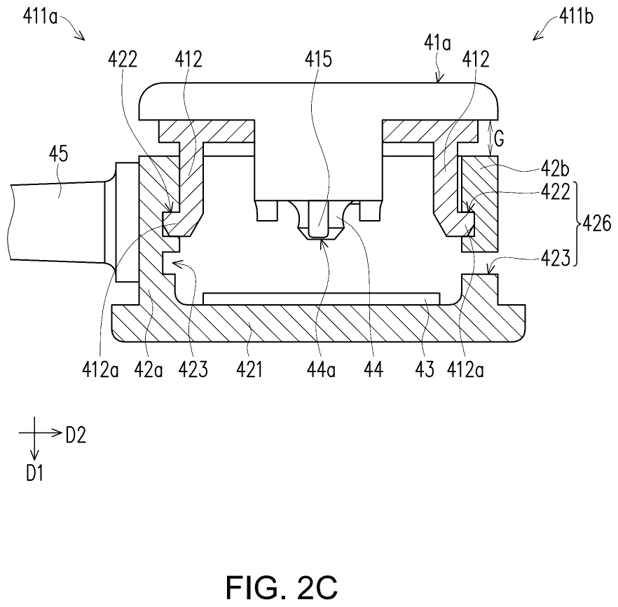

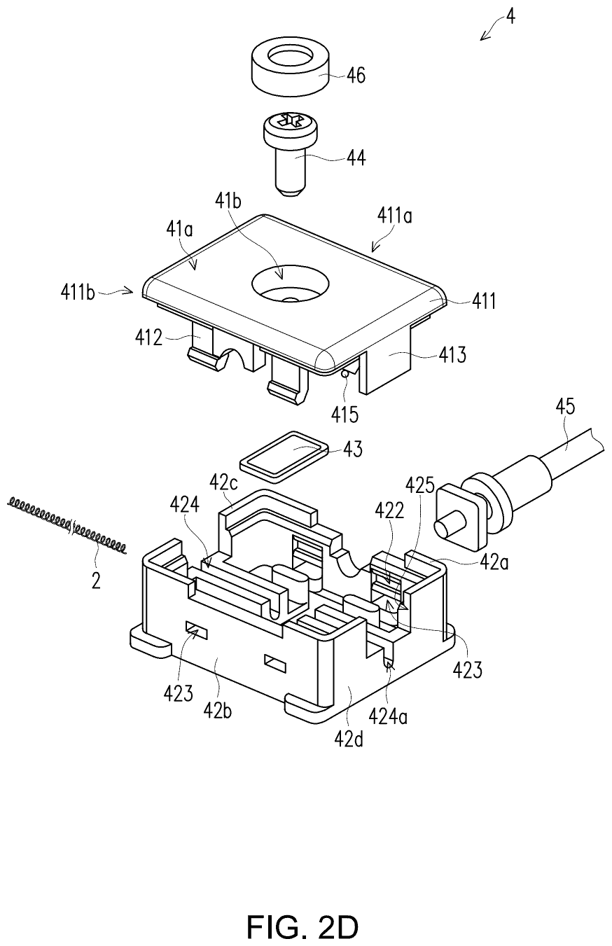

FIG. 2A is an exploded schematic view of a connector applied to the electrical stimulation system of FIG. 1, FIG. 2B is a schematic view of an upper cover and a base having a first relative position after assembling the connector of FIG. 2A, FIG. 2C is a cross-sectional view of a section A-A of FIG. 2B, and FIG. 2D is a schematic view of FIG. 2A from another perspective.

Please refer to FIG. 2A, FIG. 2B, and FIG. 2C. The connector 4 includes an upper cover 41, a base 42, an electrical pad 43, and a first fastener 44. The upper cover 41 has a top portion 411 and at least two first positioning structures 412 extending from two opposite sides of the top portion 411, wherein the upper cover 41 is adapted to cover the base 42 toward a first direction D1. The base 42 has a bottom portion 421 and at least two second positioning structures 426 respectively located on two opposite sides of the bottom portion 421, wherein each of the second positioning structures 426 has a first engaging portion 422 and a second engaging portion 423 along the first direction D1, and the first engaging portion 422 is located between the top portion 411 and the second engaging portion 423. The electrical pad 43 is disposed on the bottom portion 421 and is electrically connected to the electrical stimulation device 3 by a circuit board in the connector 4 and the external cable 45. The first fastener 44 is disposed passing through the top portion 411 and is fastened along a direction toward the bottom portion 421, wherein the first fastener 44 of the embodiment is a plastic screw, and an orthographic projection of the first fastener 44 on the bottom portion 421 of the base 42 falls within an orthographic projection range of the electrical pad 43 on the bottom portion 421 of the base 42.

When the upper cover 41 is combined with the base 42, the relative position between the upper cover 41 and the base 42 may be determined through combining the first positioning structure 412 with the first engaging portion 422 or the second engaging portion 423.

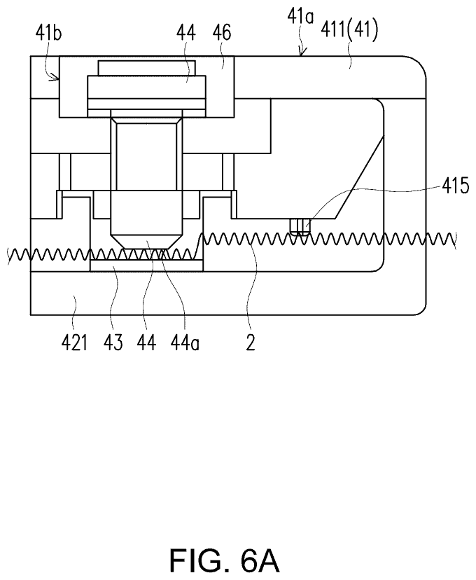

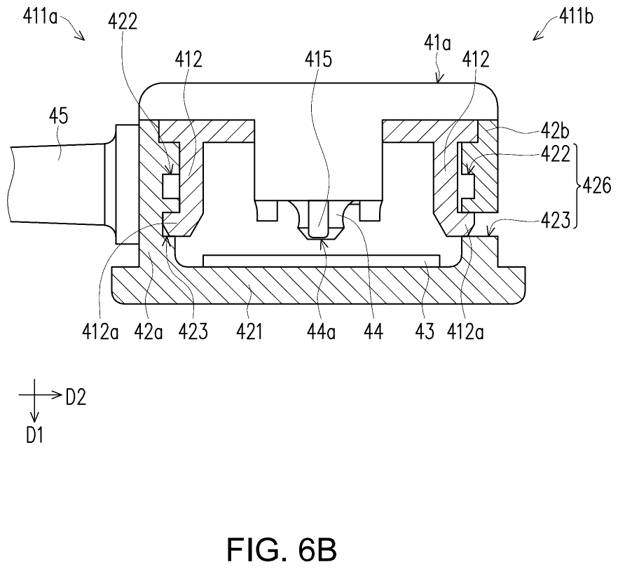

Specifically, as shown in FIG. 2C, when the first positioning structure 412 is combined with the first engaging portion 422, the upper cover 41 and the base 42 have the first relative position, and there is a gap G between the upper cover 41 and the base 42. When the first positioning structure 412 is combined with the second engaging portion 423, as shown in FIG. 6B, the upper cover 41 and the base 42 have a second relative position, and the upper cover 41 contacts the base 42.

Please refer to FIG. 2C. The first positioning structure 412 is composed of two sets of hooks 412a. The two sets of hooks 412a are formed respectively extending from a first side 411a and a second side 411b of the top portion 411 toward the first direction D1, and a hooking direction of one set of the hooks 412a extends along the second direction D2, and a hooking direction of the other set of the hooks 412a extends along the opposite direction of the second direction D2, wherein the second direction D2 is perpendicular to the first direction D1.

Please continue to refer to FIG. 2A and FIG. 2C. In addition, the second positioning structure 426 of the base 42 is formed on a first side wall 42a and a second side wall 42b connected to the bottom portion 421 of the base 42, wherein the first side wall 42a and the second side wall 42b are located on two opposite sides of the base 42. The first side wall 42a and the second side wall 42b of the base 42 respectively correspond to the first side 411a and the second side 411b of the top portion 411.

FIG. 2D is a schematic view of FIG. 2A from another perspective. Please refer to FIG. 2A, FIG. 2C, and FIG. 2D at the same time. The first engaging portion 422 of the second positioning structure 426 in the embodiment is a blind hole, the second engaging portion 423 on the first side wall 42a is a blind hole, and the second engaging portion 423 on the second side wall 42b is an open hole, but whether the first engaging portion 422 and the second engaging portion 423 are blind holes or open holes is not limited to the content of the embodiment.

In some embodiments, the first engaging portion 422 and the second engaging portion 423 may both be open holes. In the embodiment, the first engaging portion 422 and the second engaging portion 423 on the first side wall 42a are implemented with blind holes, which can reduce perforation on the appearance of the base 42 and give a user a more visually pleasing experience compared with the manner in which the first engaging portion 422 and the second engaging portion 423 are both implemented with open holes. In addition, the second engaging portion 423 on the second side wall 42b is set as an open hole for the convenience of subsequently using a hand tool to restore the upper cover 41 and the base 42 of the connector 4 from the second relative position to the first relative position and to have the gap G between the upper cover 41 and the base 42, so that the lead 2 may be disassembled from the connector 4.

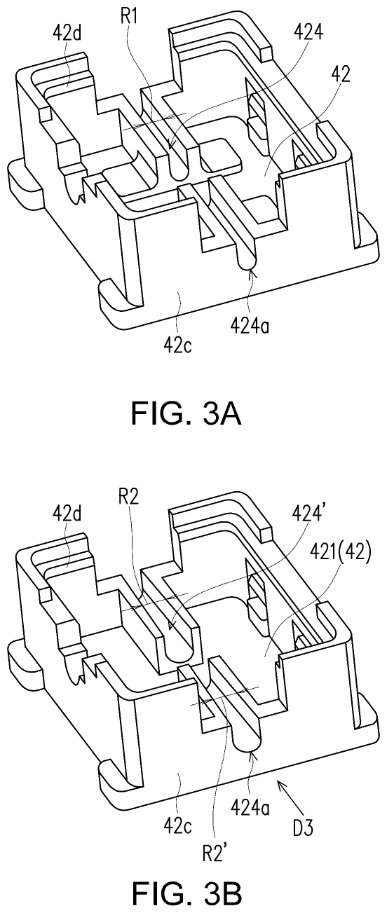

FIG. 3A is a schematic view of a base of FIG. 2A from a different perspective. Please refer to FIG. 2A, FIG. 2B, and FIG. 3A at the same time. In the embodiment, the upper cover 41 and the base 42 may be co-constructed with an opening O that allows the lead 2 to be correspondingly inserted, and the bottom portion 421 also has a lead accommodating groove 424 exposed from a side surface of the base 42 and having a recess424a, wherein the opening O is communicated with the recess424a. The lead 2 is adapted to be inserted from the opening O and then enter the base 42 via the recess424a and be accommodated in the lead accommodating groove 424.

Although in the embodiment, the upper cover 41 and the base 42 are co-constructed with the opening O, in other embodiments, the opening O may also be formed on the upper cover 41 or the base 42, which may be determined according to actual design requirements.

Specifically, the base 42 also has a third side wall 42c and a fourth side wall 42d, wherein the first side wall 42a, the second side wall 42b, the third side wall 42c, and the fourth side wall 42d are formed surrounding the bottom portion 421, and the side surfaces of the base 42 are the third side wall 42c and the fourth side wall 42d. In the embodiment, a groove diameter R1 of the lead accommodating groove 424 is fixed, but not limited thereto.

FIG. 3B is a schematic view of a variation of a lead accommodating groove of FIG. 3A. In the embodiment of FIG. 3B, groove diameters R2 and R2′ of a lead accommodating groove 424′ are variable. Specifically, in an entry direction D3 of the lead 2, the groove diameter R2′ of the lead accommodating groove 424′ gradually shrinks from the third side wall 42c toward the inside of the base 42, and the groove diameter R2 of the lead accommodating groove 424′ gradually expands from the fourth side wall 42d toward the inside of the base 42. Such arrangement manners of the groove diameters R2 and R2′ facilitate the user to pass the lead 2 through the lead accommodating groove 424′.

Please continue to refer to FIG. 2A, FIG. 2B, and FIG. 2D. The upper cover 41 also has a limiting portion 413 formed by extending from the top portion 411 along the first direction D1, and the base 42 has a limiting groove 425 corresponding to the limiting portion 413. Specifically, the limiting grooves 425 are located on the third side wall 42c and the fourth side wall 42d. The limiting groove 425 is disposed corresponding to the limiting portion 413 to limit the upper cover 41 to move relative to the base 42 only in the first direction D1 and not along the direction D2. In addition, when the upper cover 41 contacts the base 42 to have the second relative position, the limiting portion 413 may also limit the lead 2 to be in the recess424a of the lead accommodating groove 424.

In addition, as shown in FIG. 2A, the connector 4 also includes a rubber cap 46, wherein the rubber cap 46 is disposed at a void 41b of a top surface 41a of the upper cover 41, and a fixed torque screwdriver is used to pass the first fastener 44 through the rubber cap 46 and the void 41b to be locked into the upper cover 41, so that an internal flange structure of the rubber cap 46 may be used to prevent the first fastener 44 from loosening or being reversed and falling out from the upper cover 41.

Please continue to refer to FIG. 2A, FIG. 2B, and FIG. 2C. The assembly manner of the connector 4 of the embodiment is fairly simple and only requires the upper cover 41 of the connector 4 to cover the base 42 along the first direction D1 and the first positioning structure 412 to correspondingly snap into the first engaging portion 422. At this time, the upper cover 41 and the base 42 have the first relative position and there is the gap G between the upper cover 41 and the base 42.

By the way, as shown in FIG. 1 and FIG. 2A, before assembling the upper cover 41 and the base 42 together, one end 45b of the cable 45 may be electrically connected to the electrical pad 43 placed in the bottom portion 421 of the base 42. In addition, the other end 45a of the cable 45 is connected to the electrical stimulation device 3. In addition to providing electrical stimulation signals, the electrical stimulation device 3 of the embodiment may also be connected to another electronic device, such as an analyzer, a recorder, and a computer, through a wired or wireless transmission manner to analyze or record information transmitted from the connector 4.

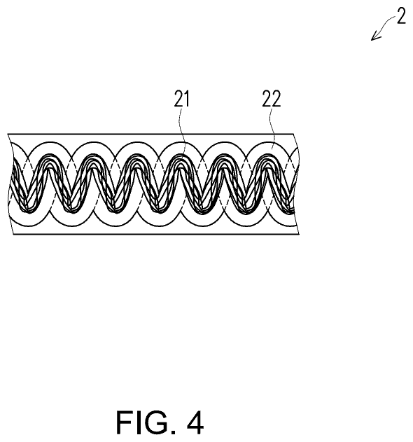

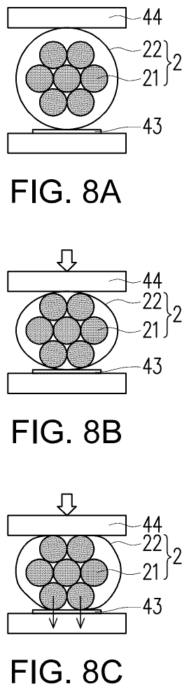

FIG. 4 is a partially enlarged schematic view of the lead. As shown in FIG. 4, the lead 2 of the embodiment is coiled and has at least one filament 21 and an insulation layer 22 covering the filament 21. It should be noted that the lead 2 of the embodiment shown in FIG. 4 is a coiled lead as an example and has multiple conductive filaments. For example, the lead 2 shown in FIG. 8A to FIG. 8C has 7 strands of filaments. In other embodiments not shown, a linear lead 2 may also be used. In addition, the lead 2 may generally refer to a lead, a cable, or other components for establishing a connection between an electrode and the electrical stimulation device 3, the lead 2 may have at least one filament 21, and the filament 21 may also include single or multiple strands. The filament 21 may also be instead of single or multiple wires, filar-wires, or filars. The connector 4 may be equipped with any thin lead 2, especially the lead 2 having the insulation layer 22. The diameter of the thin lead 2 may be less than 1.25 mm, preferably less than 0.6 mm. The distal lead 2 may be implanted through a hypodermic needle or a surgical procedure, and the proximal lead 2 may be electrically connected to the electrical stimulation device 3 via the connector 4. In addition, the electrode of the lead 2 may be a lead portion exposed by removing the insulation layer 22 and may be spiral or an additional smooth cylinder electrode.

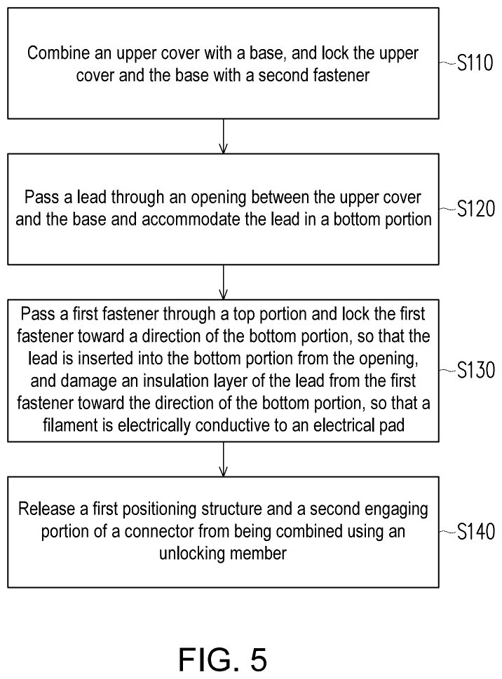

FIG. 5 is an assembly flowchart of the electrical stimulation system.

Please refer to FIG. 1 and FIG. 5 at the same time. When using the electrical stimulation system 1, a doctor first implants one end of the lead 2 into the human body, and then fixes the other end to the connector 4, so that the lead 2 is electrically conductive to the electrical stimulation device 3 through the connector 4. In addition, in order to prevent external moisture from wetting the connector 4 and affecting the electrical operation thereof, the waterproof film B may be added to cover the connector 4 and a wound for protection.

Specifically, please refer to FIG. 2A, FIG. 2B, and FIG. 5 at the same time. In step S110, the upper cover 41 is combined with the base 42, and the upper cover 41 and the base 42 are locked with a second fastener 47.

In step S120, the lead 2 is inserted into the opening O between the upper cover 41 and the base 42 and is accommodated in the bottom portion 421. More precisely, the lead 2 is inserted into the lead accommodating groove 424 via the opening O, and the lead 2 is pushed, so that the lead 2 comes out from the opening O on the other side. At this time, a part of the lead 2 is located within the range of the connector 4.

FIG. 6A is a schematic view of the lead fixed in the connector, and FIG. 6B is a schematic view of a first positioning structure combined with a second engaging portion. Please refer to FIG. 5, FIG. 6A, and FIG. 6B at the same time. In step S130, the first fastener 44 is passed through the top portion 411 and is fastened along the direction toward the bottom portion 421 (that is, the first direction D1), so that the lead 2 is inserted into the bottom portion 421 from the opening O. The insulation layer 22 of the lead 2 is damaged from the first fastener 44 toward the direction of the bottom portion 421, so that the filament 21 is electrically conductive to the electrical pad 43.

Based on the above, in the embodiment, the first fastener 44 passes through the top portion 411 to apply force to move the upper cover 41 toward the base 42 along the first direction D1, and as the first fastener 44 moves downward, the first positioning structure 412 is combined with the second engaging portion 423. At this time, the upper cover 41 and the base 42 have the second relative position, the upper cover 41 contacts the base 42, and the upper cover 41 and the base 42 fix the lead 2. At this time, a pressing surface 44a of the first fastener 44 presses against the lead 2.

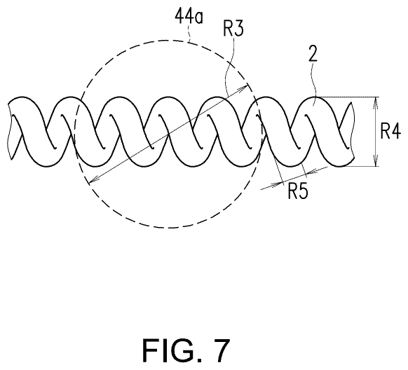

FIG. 7 is a relationship diagram of a diameter of a pressing surface of a fastener and a winding diameter of the lead. Please refer to FIG. 6A and FIG. 7 at the same time. The pressing surface 44a of the first fastener 44 is a flat surface. When the upper cover 41 contacts the base 42 and the lead 2 is fixed, the pressing surface 44a of the first fastener 44 presses against the lead 2, wherein a diameter R3 of the pressing surface 44a is greater than a winding diameter R4 (the diameter when no external force is applied) of the lead 2. In this way, the pressing surface 44a of the first fastener 44 can effectively press and fix the lead 2. The range of the winding diameter R4 (R4 refers to the outer diameter of the lead 2 after shaping if the lead 2 is not a coiled lead) is 0.3 mm to 1.2 mm. The range of a diameter R5 of the lead 2 when the lead 2 is not wound is 0.05 mm to 0.4 mm.

In addition, please refer to FIG. 2A, FIG. 6A, and FIG. 6B at the same time. The top portion 411 of the upper cover 41 also has an abutting rib 415 located between the first positioning structures 412, and when the first positioning structure 412 is combined with the second engaging portion 423, the abutting rib 415 abuts against the lead 2, wherein the abutting rib 415 abuts against the lead 2 from an upper portion of the lead 2 and is further snapped into a winding gap of the lead 2 to further prevent the lead 2 from sliding on an XY plane.

FIG. 8A to FIG. 8C are schematic views of damaging an insulation layer of the lead as the fastener is pressed downward, so that a filament is exposed and electrically contacts an electrical pad. As shown in FIG. 8A to FIG. 8C, in order for the lead 2 to be electrically conductive to the electrical pad 43, the first fastener 44 is moved deeper along the first direction D1 to contact and simultaneously press and damage the insulation layer 22 of the lead 2, so that the filament 21 of the lead 2 is exposed to physically contact the electrical pads 43 and be electrically conductive to the cable 45 through the electrical pads 43. In this way, the electrical stimulation system 1 may operate.

By the way, the first fastener 44 used to damage the insulation layer 22 of the lead 2 is made of an insulating material, which can prevent that in the case where the first fastener 44 is made of metal, when the first fastener 44 damages the insulation layer 22 to expose the filament 21, the first fastener 44 contacts the filament 21 and simultaneously forms a conductive connection with the electrical pad 43. Taking a plastic screw as the first fastener 44 as an example, the friction of the first fastener 44 against the insulation layer 22 of the lead 2 by the pressing surface 44a of the first fastener 44 is overall damage from the outer edge, instead of being performed by scraping off the insulation layer 22 using a bevel-tip structure, which can prevent damaging the filament 21.

In step S140, after the electrical stimulation system 1 is used or when the lead 2 or the connector 4 needs to be replaced, the first positioning structure 412 and the second engaging portion 423 of the connector 4 may be released from being combined using an unlocking member 5 to detach the lead 2 from the connector 4. In this way, it can be seen that the upper cover 41 may be uncapped in a simple step using the unlocking member 5.

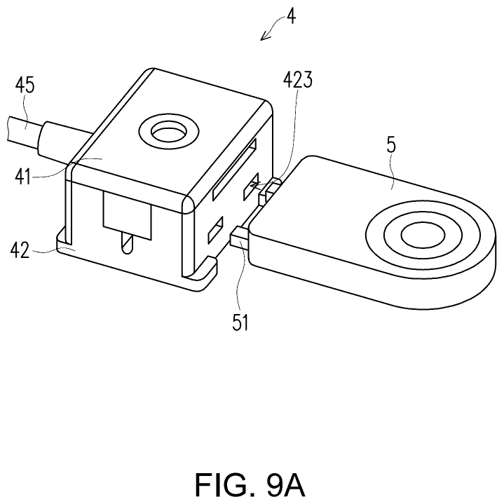

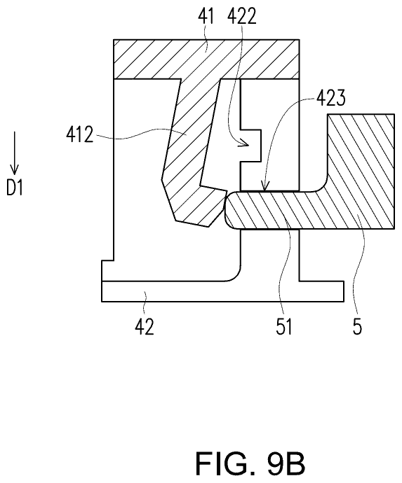

FIG. 9A is a schematic view of using an unlocking member and the connector, and FIG. 9B is a schematic view of using the unlocking member to release the upper cover and the base of the connector from being combined. Please refer to FIG. 9A and FIG. 9B at the same time. Specifically, the electrical stimulation system 1 also includes the unlocking member 5 having at least one protruding portion 51, wherein the protruding portion 51 of the unlocking member 5 cooperates with the second engaging portion 423 which is an open hole. Therefore, the protruding portion 51 may be inserted into the open hole to withdraw the first positioning structure 412 originally combined with the second engaging portion 423 from the open hole of the second engaging portion 423 to release the upper cover 41 and the second engaging portion 423 of the base 42 from being combined.

Please refer to FIG. 9B and FIG. 2C at the same time. After releasing the first positioning structure 412 and the second engaging portion 423 from being combined, an elastic restoring force of the first positioning structure 412 displaces the upper cover 41 relative to the base 42 along an opposite direction of the first direction D1, and the first positioning structure 412 is correspondingly snapped into the first engaging portion 422 again. At this time, the lead 2 may be pulled out from the connector 4, so that the connector 4 and the lead 2 are separated.

### Second Embodiment

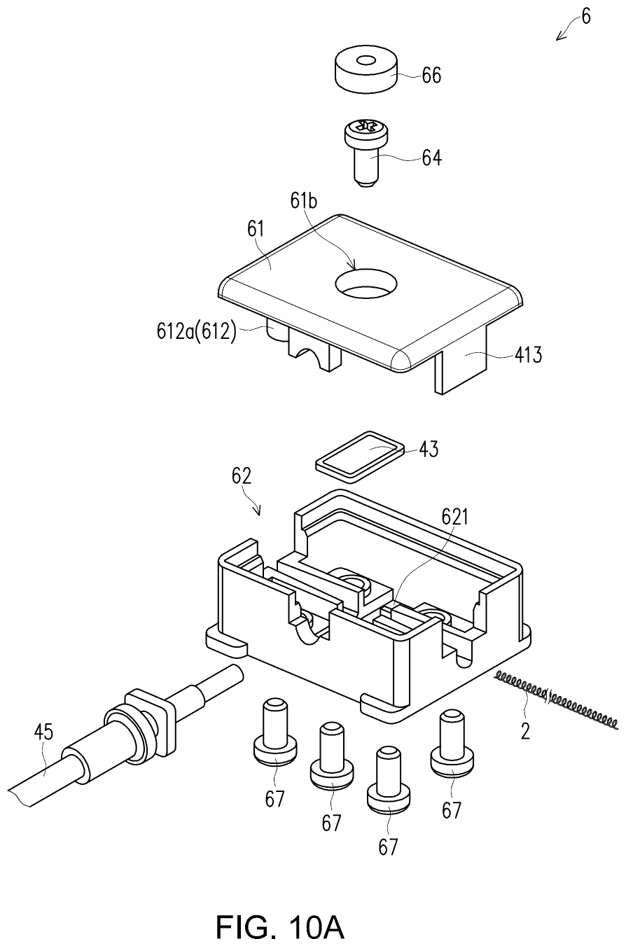

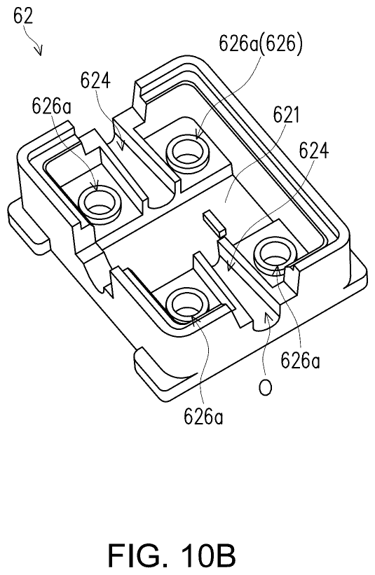

FIG. 10A is an exploded schematic view of a connector of an electrical stimulation system according to a second embodiment of the disclosure, and FIG. 10B is a schematic view of a base in FIG. 10A from another perspective. Please refer to FIG. 10A and FIG. 10B at the same time. The difference between the embodiment and the first embodiment is that in the first embodiment, the first positioning structure 412 disposed on the upper cover 41 is hooked with one of the engaging portions of the second positioning structure 426 disposed on the base 42 to assemble the upper cover 41 and the base 42 together. However, in the embodiment, the upper cover 61 and the base 62 are locked together through a second fastener 67, without the need for the original hooking structure.

Specifically, a first positioning structure 612 disposed on an upper cover 61 is a shank 612a, a second positioning structure 626 disposed on a base 62 is a screw hole 626a, and the position of the shank 612a corresponds to the position of the screw hole 626a, wherein the second fastener 67 is passed through the corresponding screw hole 626a and the shank 612a is locked to combine the upper cover 61 and the base 62 together.

In the embodiment, the second fastener 67 is passed through a bottom portion 621 from the bottom of the base 62 to be locked to the upper cover 61, but not limited thereto. In other embodiments not shown, the upper cover 61 may be provided with the screw hole 626a and the base 62 may be provided with the shank 612a, and the second fastener 67 is passed through the screw hole 626a from above the base 62 to be locked to the shank 612a disposed on the base 62.

Incidentally, although 4 second fasteners 67 are shown in FIG. 10A, the disclosure is not limited thereto. In the case of an appropriate structural design, only 1 second fastener 67 may be used to achieve the purpose of ensuring that the upper cover 61 securely covers the base 62.

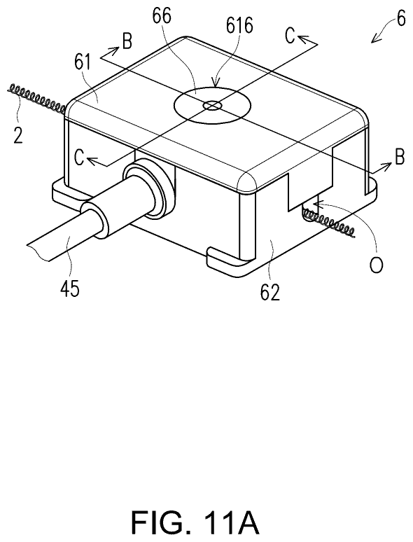

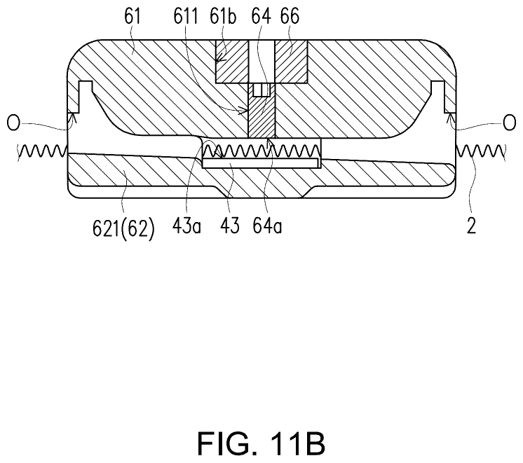

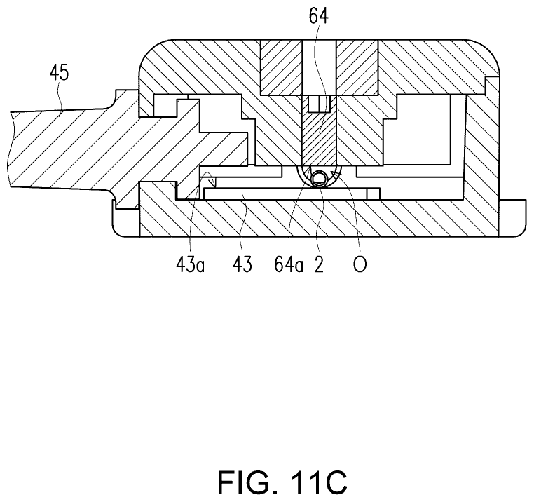

FIG. 11A is an assembly schematic view of a connector of FIG. 10A, FIG. 11B is a cross-sectional view along a sectional line B-B of FIG. 11A, and FIG. 11C is a cross-sectional view along a sectional line C-C of FIG. 11A.

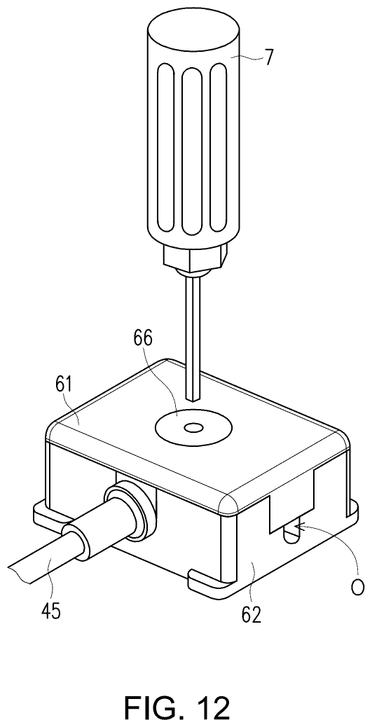

Please refer to FIG. 10A and FIG. 11A at the same time. A connector 6 of the embodiment also includes a rubber cap 66 disposed in a void 61b of the upper cover 61, and a first fastener 64 is correspondingly passed through the rubber cap 66 and is placed into a pillar hole 611 of the upper cover 61 via a hand tool 7 (shown in FIG. 12), such as a screwdriver.

As shown in FIG. 10A, FIG. 10B, and FIG. 11A, after the upper cover 61 and the base 62 are combined together, openings O are formed on two opposite sides of the connector 6, wherein the openings O are formed on the base 62, but not limited thereto. The openings O may also be formed on the upper cover 61 or may be co-constructed by the upper cover 61 and the base 62. The lead 2 is inserted into the connector 6 via the opening O.

Please refer to FIG. 10A, FIG. 11A, and FIG. 11B at the same time. The lead 2 entering from the opening O on one side of the connector 6 comes out of the connector 6 from the opening O on the other side of the connector 6 via the guidance and positioning of a lead accommodating groove 624.

As shown in FIG. 11A, FIG. 11B, and FIG. 11C, a pressing surface 64a of the first fastener 64 is separated from an upper surface 43a of the electrical pad 43 by a distance, and the lead 2 inserted into the connector 6 is located between the upper surface 43a of the electrical pad 43 and the pressing surface 64a of the first fastener 64.

By adjusting the position of the first fastener 64 in the pillar hole 611, the relative distance between the first fastener 64 and the bottom portion 621 of the base 62 can be changed, so that the lead 2 is electrically conductive to the electrical pad 43.

FIG. 12 is a schematic view of using the hand tool 7 to adjust a position of a first fastener in a pillar hole, wherein the hand tool 7 is a fixed torque screwdriver as an example, so that a pressing surface of the first fastener 64 presses against the lead 2 and damages an insulation layer of the lead 2, so that a filament is electrically conductive to an electrical pad of the base 62.

Incidentally, in the embodiment, there are still structures or arrangements that are not mentioned but are explained in the first embodiment, such as the limiting portion 413 and the abutting rib 415 of an upper cover, the connection manner between the cable 45 and the electrical pad 43 . . . , and the structures and the arrangements may be changed in design according to actual requirements.

In summary, in the connector, the electrical stimulation system using the connector, and the assembly method of the electrical stimulation system of the disclosure, the first fastener is passed through the upper cover and is fastened along the direction toward the base to damage the insulation layer of the lead, so that the filament may be electrically conductive to the electrical pad of the connector via a simple damage step, thereby enabling the lead to be electrically conductive to the electrical stimulation device.

What is claimed is:

1. A connector, adapted to fix a lead and to electrically connect the lead to an electrical stimulation device, wherein the lead comprises a filament and an insulation layer covering the filament, the connector comprising:
an upper cover, having a top portion;
a base, combined with the upper cover and having a bottom portion, wherein at least one of the upper cover and the base forms an opening;
an electrical pad, disposed on the bottom portion and electrically connected to the electrical stimulation device; and
a first fastener, passed through the top portion and fastened along a direction toward the bottom portion,
wherein when the lead is inserted into the connector from the opening, the first fastener moves along the direction toward the bottom portion to damage the insulation layer of the lead, so that the filament is electrically conductive to the electrical pad.

2. The connector according to claim 1, further comprising at least one second fastener to fasten the upper cover and the base.

3. The connector according to claim 1, wherein the bottom portion has a lead accommodating groove exposed from a side surface of the base and having a recess.

4. The connector according to claim 3, wherein the upper cover also has a limiting portion, the limiting portion extends from the top portion along a first direction, the base has a plurality of limiting grooves located on two opposite sides, and the limiting grooves are combined with the corresponding limiting portions to form the opening with the recess.

5. The connector according to claim 3, wherein along an entry direction of the lead, a groove diameter of the lead accommodating groove varies.

6. The connector according to claim 1, wherein a bottom portion of the first fastener has a pressing surface, and a diameter of the pressing surface is greater than a winding diameter of the lead.

7. The connector according to claim 1, further comprising:
at least two first positioning structures extending from two opposite sides of the top portion of the upper cover; and
at least two second positioning structures, respectively located on two opposite sides of the bottom potion, wherein each of the second positioning structures has a first engaging portion and a second engaging portion along a first direction, and the first engaging portion is located between the top portion and the second engaging portion,
wherein when the first positioning structures are combined with the first engaging portions, there is a gap between the upper cover and the base, and the upper cover and the base have a first relative position,
wherein when the first positioning structures are combined with the second engaging portions, the upper cover is combined with the base, and the upper cover and the base have a second relative position.

8. The connector according to claim 7, wherein the first positioning structure has two sets of hooks, and the two sets of hooks are formed respectively extending from a first side and a second side of the top portion toward the first direction, wherein hooking directions of the two sets of hooks are opposite.

9. The connector according to claim 7, wherein the top portion also has an abutting rib located between the first positioning structures, and when the first positioning structures are combined with the second engaging portions, the abutting rib abuts against the lead.

10. An electrical stimulation system, comprising:
a lead, comprising a filament and an insulation layer covering the filament;
an electrical stimulation device;
a connector, adapted to fix the lead and to electrically connect to the electrical stimulation device, the connector comprising:
an upper cover, having a top portion;
a base, combined with the upper cover and having a bottom portion, wherein at least one of the upper cover and the base forms an opening, and the lead is inserted into the connector from the opening;
an electrical pad, disposed on the bottom portion and electrically connected to the electrical stimulation device; and
a first fastener, passed through the top portion and fastened along a direction toward the bottom portion, wherein the first fastener moves along the direction toward the bottom portion to damage the insulation layer of the lead, so that the filament is electrically conductive to the electrical pad.

11. The electrical stimulation system according to claim 10, wherein the connector further comprises at least one second fastener to fasten the upper cover and the base.

12. The electrical stimulation system according to claim 10, wherein the connector further comprises:
at least two first positioning structures extending from two opposite sides of the top portion of the upper cover; and
at least two second positioning structures, respectively located on two opposite sides of the bottom potion, wherein each of the second positioning structures has a first engaging portion and a second engaging portion along a first direction, and the first engaging portion is located between the top portion and the second engaging portion,
wherein when the first positioning structures are combined with the first engaging portions, there is a gap between the upper cover and the base, and the upper cover and the base have a first relative position,
wherein when the first positioning structures are combined with the second engaging portions, the upper cover is combined with the base, and the upper cover and the base have a second relative position.

13. The electrical stimulation system according to claim 12, further comprising an unlocking member having at least one protruding portion, wherein the second engaging portion is an open hole, and the protruding portion of the unlocking member cooperates with the open hole.

14. The electrical stimulation system according to claim 10, wherein the bottom portion has a lead accommodating groove exposed from a side surface of the base and having a groove.

15. The electrical stimulation system according to claim 10, wherein a bottom portion of the first fastener has a pressing surface, and a diameter of the pressing surface is greater than a winding diameter of the lead.

16. An assembly method of an electrical stimulation system, comprising:
providing a lead, comprising a filament and an insulation layer covering the filament;
providing an electrical stimulation device; and
providing a connector, adapted to fix the lead and comprising an upper cover, a base, an electrical pad, and a first fastener, wherein the upper cover has a top portion, the upper cover is combined with the base, the base has a bottom portion, at least one of the upper cover and the base forms an opening, the lead is inserted into the connector from the opening, and the electrical pad is disposed on the bottom portion and is electrically connected to the electrical stimulation device,
wherein when the first fastener is passed through the top portion and is fastened along a direction toward the bottom portion, the first fastener moves along the direction toward the bottom portion to damage the insulation layer of the lead, so that the filament is electrically conductive to the electrical pad, and the lead is electrically connected to the electrical stimulation device.

17. The assembly method of the electrical stimulation system according to claim 16, wherein the connector further comprises at least two first positioning structures extending from two opposite sides of the top portion of the upper cover; and at least two second positioning structures respectively located on two opposite sides of the bottom portion, wherein each of the second positioning structures has a first engaging portion and a second engaging portion along a first direction, and the first engaging portion is located between the top portion and the second engaging portion, the assembly method of the electrical stimulation system comprising:
combining the first positioning structures with the first engaging portions, wherein there is a gap between the upper cover and the base, and the upper cover and the base have a first relative position,
combining the first positioning structures with the second engaging portions, wherein the upper cover covers the base, and the upper cover and the base have a second relative position.

18. The assembly method of the electrical stimulation system according to claim 17, further comprising:
correspondingly inserting a protruding portion of an unlocking member into the second engaging portion to release the first positioning structure and the second engaging portion from being combined.
